(19) United States
(12) Patent Application Publication
(10) Pub. No.: US 2022/0391010 A1
(43) Pub. Date: Dec. 8, 2022

(54) INFORMATION PROCESSING SYSTEM, INFORMATION PROCESSING DEVICE, INFORMATION PROCESSING METHOD, AND RECORDING MEDIUM

(71) Applicant: NEC Corporation, Minato-ku,, Tokyo (JP)

(72) Inventors: Kenichiro FUKUSHI, Tokyo (JP); Hiroaki Nakano, Tokyo (JP); Toshinori TAKEMURA, Tokyo (JP); Kentaro NAKAHARA, Tokyo (JP); Hannah POKKA, Tokyo (JP); Itsumi KATO, Tokyo (JP); Akira KAMEI, Tokyo (JP); Chenhui HUANG, Tokyo (JP); Hiroshi KAJITANI, Tokyo (JP); Zhenwei WANG, Tokyo (JP); Koichi MORIKAWA, Tokyo (JP); Hiroshi OKUDA, Tokyo (JP)

(73) Assignee: NEC Corporation, Minato-ku,, Tokyo (JP)

(21) Appl. No.: 17/769,827

(22) PCT Filed: Oct. 31, 2019
(86) PCT No.: PCT/JP2019/042807

§ 371 Date: Apr. 18, 2022

(51) Int. Cl. G06F 3/01

(52) U.S. Cl. CPC G06F 3/011

(57) ABSTRACT

An information processing system includes an acquisition unit configured to acquire sensing data from a sensor device used for a foot, and a determination unit configured to determine an attached state of the sensor device to the foot according to a motion of the foot calculated using the sensing data.

### TECHNICAL FIELD

The present invention relates to an information processing system, an information processing device, an information processing method, and a recording medium.

### BACKGROUND ART

Technologies as to how to grasp motions of feet have been developed.

For example, Patent Document 1 discloses a motion analysis device configured to determine a first period including a period of time to place a user's right foot on the ground and a second period including a period of time to place a user's left foot on the ground by use of an inertial measurement unit attached to a user's waist.

### CITATION LIST

### Patent Literature Document

Patent Document 1: Japanese Patent Application Publication No. 2016-32611

### SUMMARY OF INVENTION

### Technical Problem

When sensors configured to measure motions of feet are inappropriately attached to feet, sensors may produce data to be thought of as inappropriate data.

An exemplary object of the present invention is to provide an information processing system, an information processing device, an information processing method, and a recording medium, which can solve the aforementioned problem.

### Solution To Problem

In a first aspect of the present invention, an information processing system includes an acquisition unit configured to acquire sensing data from a sensor device used for a foot, and a determination unit configured to determine an attached state of the sensor device to the foot according to a motion of the foot calculated using the sensing data.

In a second aspect of the present invention, an information processing device includes an acquisition unit configured to acquire sensing data from a sensor device used for a foot, and a determination unit configured to determine an attached state of a sensor device to the foot based on a motion of the foot calculated using the sensing data.

In a third aspect of the present invention, an information processing method includes the steps of: acquiring sensing data from a sensor device used for a foot; and determining an attached state of the sensor device to the foot according to a motion of the foot calculated using the sensing data.

In a fourth aspect of the present invention, a storage medium is configured to store a program causing a computer to execute the steps of: acquiring sensing data from a sensor device used for a foot; and determining an attached state of the sensor device to the foot according to a motion of the foot calculated using the sensing data.

### Advantageous Effects of Invention

According to the information processing system, the information processing device, the information processing method, and the recording medium described above, it is possible to determine attached states of sensors.

### BRIEF DESCRIPTION OF DRAWINGS

FIG. 1 is a schematic diagram showing an exemplary device configuration of an information processing system according to the exemplary embodiment.

FIG. 2 is a block diagram showing an exemplary functional configuration of an information processing device according to the exemplary embodiment.

FIG. 3 is a schematic illustration showing a setting example of a coordinates system used to translate sensing data of sensor devices according to the exemplary embodiment.

FIG. 4 is a graph showing a trace of a left foot of a target person.

FIG. 5 is a flowchart showing an example of a procedure to determine whether a sensor device is attached to a left foot or a right foot via the information processing device according to the exemplary embodiment.

FIG. 6 is a bottom view showing a shape example of the sensor device in its lower face according to the exemplary embodiment.

FIG. 7 is an upper view showing a shape example of the sensor device in its upper face according to the exemplary embodiment.

FIG. 8 is a side view showing a shape example of the sensor device in its side face according to the exemplary embodiment.

FIG. 9 is a block diagram showing another functional configuration of the information processing system according to another exemplary embodiment.

FIG. 10 is a block diagram showing another functional configuration of the information processing device according to another exemplary embodiment.

FIG. 11 is a flowchart showing an example of a procedure for an information processing method according to another exemplary embodiment.

FIG. 12 is a block diagram showing an exemplary configuration of a computer according to at least one exemplary embodiment.

### DESCRIPTION OF EXEMPLARY EMBODIMENTS

The exemplary embodiment of the present invention will be described below, however, the exemplary embodiment described below may not necessarily limit the scope of the invention as defined in the appended claims. In addition, not all the combinations of features described in the exemplary embodiment may be essential to solutions of the invention.

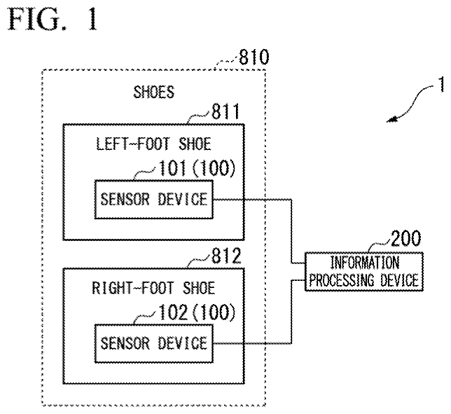

FIG. 1 is a schematic diagram showing an exemplary device configuration of an information processing system according to the exemplary embodiment. According to the configuration shown in FIG. 1, an information processing system 1 includes sensor devices 100 and an information processing device 200.

The information processing system 1 is a system configured to measure and analyze motions of human feet such as gait. The information processing system 1 is configured to determine an attached state of a sensor (i.e., the sensor device 100) used to measure a foot motion. When a sensor is inappropriately attached to a foot, sensing data (or sensor measurements) would be thought of as inappropriate data, and therefore results of calculations using sensing data would be thought of as inappropriate results. In this connection, when the information processing system 1 is configured to determine an attached state of a sensor, it is possible prevent inappropriate sensing data and inappropriate results of calculations.

The sensor devices 100 are attached to shoes worn by a person subject to analysis using the information processing system 1. For example, holes to be engaged with the sensor devices 100 may be formed on the upper faces of bodies of shoes beneath insoles. A workman who attempts to attach the sensor devices 100 to the shoes may temporarily remove the insoles from the shoes, put the sensor devices 100 into the holes of shoes, and then place the insoles inside the shoes again.

Alternatively, it is possible to form holes for engaging with the sensor devices 100 in the insoles of the shoes (e.g., backsides of shoes). A workman who attempts to place the sensor devices 100 in the shoes may temporarily remove the insoles from the shoes, put the sensor devices 100 into the holes of the insoles, and thereafter place the insoles inside the shoes again.

For example, the sensor device 100 may include an IMU (Inertial Measurement Unit) which is configured to measure triaxial acceleration and triaxial angular velocity by itself, thus measuring the motion of the sensor device 100 on its own. In addition, the sensor device 100 may include a communication unit configured to transmit sensing data to the information processing device 200.

The motions of the sensor devices 100 to be measured by the sensor devices 100 by themselves can be regarded as equivalents of motions of shoes equipped with the sensor devices 100. In addition, the motions of the sensor devices 100 to be measured by the sensor devices 100 by themselves can be regarded as equivalents of motions of feet of a person which will be analyzed by the information processing system 1.

Hereinafter, a person subject to analysis by the information processing system 1 will be referred to as a target person. In this connection, the feet of a target person will be simply referred to as feet.

Shoes 810 are denoted as shoes equipped with the sensor devices 100. In a pair of shoes 810, a shoe for a left foot will be referred to as a left-foot shoe 811 while a shoe for a right foot will be referred to as a right-foot shoe 812.

The following examples refer to the situation in which the sensor devices 100 are attached to the left-foot shoe 811 and the right-foot shoe 812. In this connection, the sensor device 100 attached to the left-foot shoe 811 will be referred to as a sensor device 101 while the sensor device 100 attached to the right-foot shoe 812 will be referred to as a sensor device 102.

When the information processing system 1 is designed to simply measure a motion of a foot, however, the sensor device 100 needs to be attached to either the left-foot shoe 811 or the right-foot shoe 812.

The method for attaching the sensor devices 100 to body parts should not be necessarily limited to a method to attach the sensor devices 100 to shoes since the present exemplary embodiment may adopt any attaching methods which can measure motions of feet. For example, it is possible to fix the sensor devices 100 to feet by use of bands or the like.

The information processing device 200 is configured to acquire sensing data produced by the sensor devices 100. The information processing device 200 is configured to analyze motions of feet of a target person using the sensing data. In addition, the information processing device 200 is configured to determine an attached state of the sensor device(s) 100 using the sensing data.

For example, the information processing device 100 may be configured of a smartphone. Since a target person carries the information processing device 200 which is configured of a handheld-type device such as a smartphone, it is assumed that the information processing device 200 may be normally located in proximity to the sensor devices 100. Accordingly, it is possible to carry out a communication between the information processing device 100 and the sensor devices 100 with ease, for example, the information processing device 200 may communicate with the sensor devices 100 via near-field communication.

In this connection, the information processing device 200 can be configured of other types of devices than smartphones, e.g., personal computers (PC).

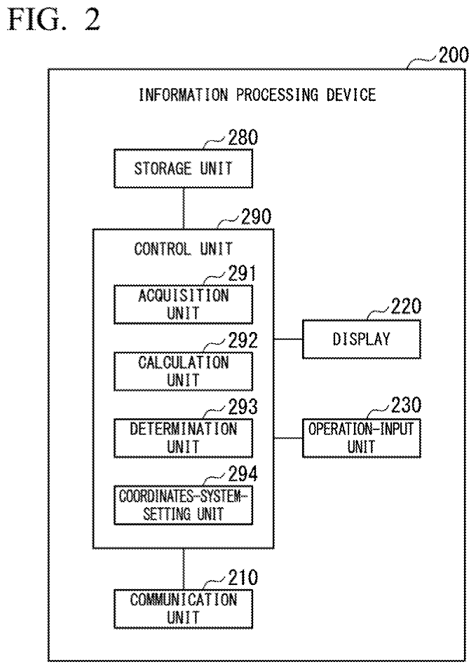

FIG. 2 is a block diagram showing an exemplary functional configuration of the information processing device 200. According to the configuration shown in FIG. 2, the information processing device 200 includes a communication unit 210, a display 220, an operation-input unit 230, a storage unit 280, and a control unit 290. The control unit 290 further includes an acquisition unit 291, a calculation unit 292 a determination unit 293, and a coordinates-system-setting unit 294.

The communication unit 210 is configured to communicate with other devices. In particular, the communication unit 210 communicates with the sensor devices 100 to receive sensing data from the sensor devices 100.

A communication method of the communication unit 210 is not necessarily limited to a specific method as long as the communication unit 210 can perform communication with the sensor devices 100.

For example, the display 220 includes a display screen such as a liquid-crystal panel or an LED (Light-Emitting Diode) panel to display various kinds of images. For example, the display 220 is configured to display analysis results produced by the information processing device 200 to analyze sensing data of the sensor devices 100.

The display 220 may serve as an example of an alarm output unit. When the information processing device 200 determines an inappropriately-attached state of the sensor device(s) 100, the display 220 may display an alarm message indicating an inappropriately-attached state of the sensor device(s) 100.

In this connection, the method for the information processing device 200 to output an alarm is not necessarily limited to the aforementioned method for displaying an alarm message. For example, the information processing device 200 is equipped with a speaker to output an alarm sound or an alarm-message speech instead of or in addition to an alarm message displayed on the screen, alternatively, the information processing device 200 may output combinations of alarm display and alarm sound.

For example, the operation-input unit 230 is equipped with an input device such as a touch sensor configuring a touch panel embedded with the display screen of the display 220 so as to receive a user operation. For example, the operation-input unit 230 may receive a user operation instructing analysis of sensing data output from the sensor devices 100.

The storage unit 280 is configured to store various types of data. The storage unit 280 is configured using a storage device installed in the information processing device 200.

The control unit 290 is configured to carry out various types of control by controlling various parts of the information processing device 200. It is possible to achieve the function of the control unit 290 when a CPU (Central Processing Unit) installed in the information processing device 200 reads and executes programs from the storage unit 280.

The acquisition unit 291 is configured to acquire sensing data produced by the sensor devices 100. The acquisition unit 291 is configured to extract sensing data from the received data of the communication unit 210 received from the sensor devices 100.

The calculation unit 292 is configured to calculate motions of feet of a target person when walking on roads using sensing data output from the sensor devices 100.

For example, the calculation unit 292 may calculate traces of feet of a target person when walking on roads by use of sensing data output from the sensor devices 100. For example, the calculation unit 292 is configured to integrate acceleration vectors of feet indicated by sensing data so as to produce velocity vectors of feet, thus calculating traces of feet.

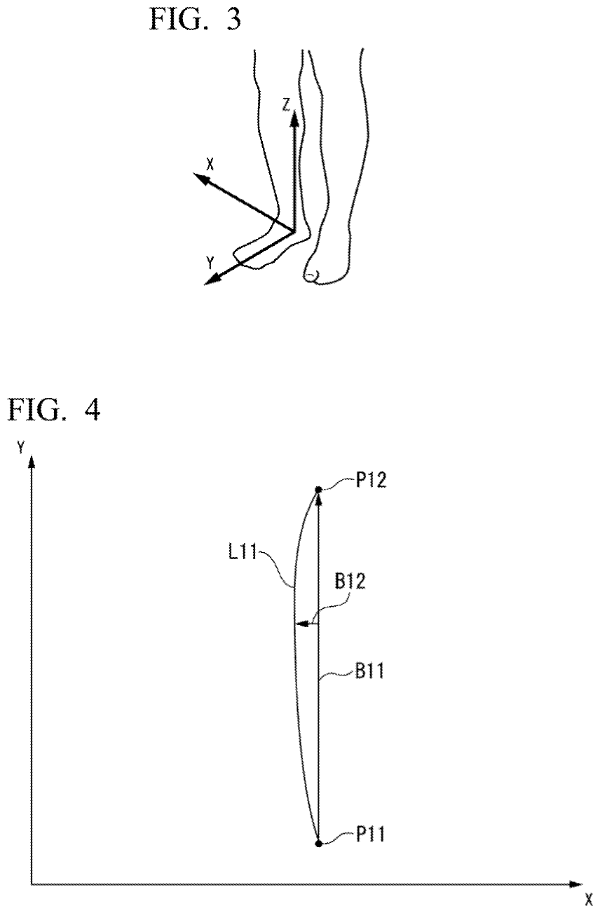

To reduce an influence of vertical motions of feet, the calculation unit 292 may extract components of acceleration of feet in a horizontal plane based on angular velocity indicated by sensing data. For example, components in a horizontal plane may correspond to X-coordinate components and Y-coordinate components described later in conjunction with FIG. 3.

The calculation unit 292 may calculate an offset of sensing data so as to correct sensing data by removing the offset. Herein, the offset is a steady-state deviation between an actual value and a measured value, which will be referred to as a bias. For example, an offset value may appear in sensing data produced by the sensor device(s) 100 in a statical condition.

In addition, the calculation unit 292 may perform a process to correct a drift of sensing data due to an integral calculus.

In this connection, it is possible to use the existing correction methods as the method for the calculation unit 292 to correct a bias of sensing data and the method for the calculation unit 292 to correct a drift of sensing data.

The calculation unit 292 is configured to calculate a trace of a foot and to determine which of a left foot and a right foot is equipped with the sensor device 100 based on the trace of a foot. This feature will be explained with reference to FIGS. 3 and 4.

FIG. 3 is a schematic illustration showing a setting example of a coordinates system used to translate sensing data produced by the sensor device(s) 100.

In an example of FIG. 3, an X axis is set in a rightward-leftward direction in view of a target person such that a positive side of the X axis represents a rightward direction of a target person. In addition, a Y axis is set in a forward-backward direction in view of a target person such that the positive side of the Y axis represents a forward direction of a target person. Moreover, a Z axis is set in an upward-downward direction in view of a target person such that the positive side of the Z axis represents an upward direction of a target person.

FIG. 4 is a graph showing an example of a trace of a left foot of a target person.

FIG. 4 shows an example in which a target person is walking forwards in the coordinates system which is described above with reference to FIG. 3. That is, FIG. 4 shows an example in which a target person is walking in a positive direction on the Y axis. In this connection, the X axis indicates a rightward-leftward direction of a target person, wherein the positive side of the X axis represents a rightward direction.

A line segment L11 shows an example of a trace of a left foot in which its heel leaves from the ground at a point P11 and then touches down to the ground again at a point P12. An arrow B11 is directed from the point P11 to the point P12. The direction of the arrow B11 substantially matches the positive direction on Y-axis coordinates representing a traveling direction of walking.

Comparing the line segment L11 with the arrow B11, the line segment L11 is partially expanded from the arrow B11 in its left side. An arrow B12 is an arrow showing an example of a deviation of the line segment L11 compared to the arrow B11, wherein the arrow B12 is directed leftwards (i.e., a negative direction on X-axis coordinates).

As described above, the trace of a left foot on a walk may be greatly shifted leftwards in a traveling direction, and then a deviation will be gradually reduced. That is, the trace of a left foot is shaped like a leftward-swollen arc as the line segment L11.

In the case of a right foot, its trace will be greatly shifted rightwards in a traveling direction, and then a deviation will be gradually reduced. That is, the trace of a right foot on a walk is shaped like a rightward-swollen arc. The motion of a foot to be shifted leftwards or rightwards such that the trace of a foot on a walk is shaped like an arc shape will be referred to as a circumduction motion.

As described above, a left foot and a right foot have different traces of walking in a leftward-deviating direction and a rightward-deviating direction. That is, the trace of a left foot is shifted leftwards while the trace of a right foot is shifted rightwards. By determining a deviating direction of a foot, it is possible to determine which of a left foot and a right foot is equipped with the sensor device 100.

For this reason, the calculation unit 292 is configured to calculate the motion of a foot including a lateral deviation of a foot in a traveling direction of walking. Next, the determination unit 293 described later is configured to determine which of a left foot and a right foot is equipped with the sensor device 100 based on calculation results.

The determination unit 293 is configured to determine an attached state of the sensor device 100 to a foot of a target person based on the motion of a foot calculated using sensing data produced by the sensor device 100. As described above, the sensor devices 100 are attached to the shoes 810; hence, the sensor devices 100 are attached to feet when a target person wears the shoes 810.

For example, the determination unit 293 is configured to determine any one or more of the following items.
(1) An upward-downward direction of the sensor device 100

For Example, the determination unit 293 determines an upward-downward direction of the sensor device 100 based on acceleration of gravity detected by the sensor device 100.
(2) A forward-backward direction of the sensor device 100

For example, the determination unit 293 determines a traveling direction of walking as a forward direction based on a trace of a foot on a walk calculated by the calculation unit 292.
(3) A rightward-leftward direction of the sensor device 100

For example, the determination unit 293 determines a rightward-leftward direction of the sensor device 100 based on the upward-downward direction and the upward-downward direction of the sensor device 100.
(4) A right/left foot equipped with the sensor device 100

For example, the determination unit 293 determines which of a left foot and a right foot is equipped with the sensor device 100 based on the circumduction motion in a trace of a foot on a walk calculated by the calculation unit 292. When the circumduction motion indicates a trace of a foot to be swollen leftwards in a traveling direction, the determination unit 293 determines that the sensor device 100 is attached to a left foot. When the circumduction motion indicates a trace of a foot to be swollen rightwards in a traveling direction, the determination unit 293 determines that the sensor device 100 is attached to a right foot.

The determination unit 293 may determine the appropriateness of an attached state of the sensor device 100 to a foot of a target person. Specifically, the determination unit 293 may determine whether the aforementioned items (1) through (4) match a presumed direction and a distinction of left/right feet. That is, the determination unit 293 may determine whether or not the sensor device 100 is attached to a predetermined foot as instructed when the direction of the sensor device 100 to be attached to either a left foot or a right foot is determined in advance.

Upon determining that any one or more of the aforementioned items (1) through (4) do not match the presumed direction and a distinction of a left/right feet, the determination unit 293 may control the display 220 to display an alarm message representing the determination result on the screen.

For example, it is possible to display on the display 220 an alarm message representing a content of correction such as “please exchange a left-shoe sensor and a right-shoe sensor” or “please fix a left-shoe sensor in a vertically-reversed manner”.

When the coordinates-system setting unit 294 sets a coordinates system described later, the determination unit 293 may display on the display 220 the content of the detected malfunction and a message indicating an auto-correction in addition to or instead of an alarm message. For example, the determination unit 293 may display on the display 220 a message such as “upon detecting a left-shoe sensor attached in a vertically-reversed manner, sensor measurements will be recalculated in a vertically-reversed manner”.

The coordinates-system-setting unit 294 is configured to set a coordinates system to translate the sensing data of the sensor device 100 based on an attached state of the sensor device 100 to a foot of a target person. For example, the coordinates-system-setting unit 294 may perform coordinates conversion such as affine transformation of sensing data of the sensor device 100 to adjust sensing-data values to a predetermined coordinates system.

According to the determination results relating to the aforementioned items (1) through (4), for example, the coordinates-system-setting unit 294 may set a distinction of left/right feet to be equipped with the sensor device 100, thus setting coordinates in an upward-downward direction, a forward-backward direction, and a rightward-leftward direction.

As described above, FIG. 3 shows an example in which X-axis coordinates correspond to rightward-leftward coordinates, i.e., the positive side of an X axis corresponding to a rightward direction. In addition, Y-axis coordinates correspond to forward-backward coordinates, i.e., the positive side of a Y axis corresponding to a forward direction. The coordinates-system-setting unit 294 may set a coordinates system similar to the coordinates system of FIG. 3.

In this connection, the coordinates-system-setting unit 294 is not an essential part of the information processing device 200. When the information processing device 200 precludes the coordinates-system-setting unit 294, as described above, the display 220 may display an alarm message on the screen and urge a user (e.g., a target person) of the information processing system 1 to correct the attached state of the sensor device 100.

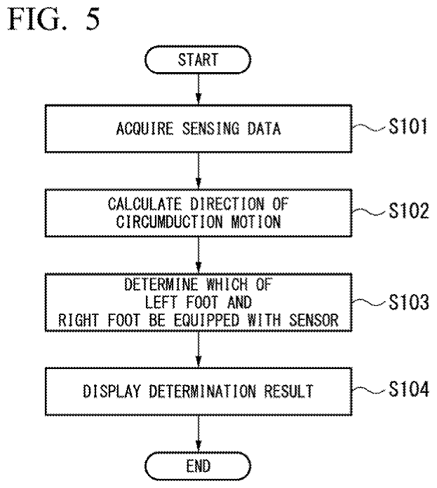

Next, the operation of the information processing device 200 will be described with reference to FIG. 5.

FIG. 5 is a flowchart showing an example of a procedure to determine a distinction of a left foot and a right foot to be equipped with the sensor device 100.

According to the procedure of FIG. 5, the acquisition unit 291 acquires sensing data of the sensor device 100 (step S101).

Next, the calculation unit 292 calculates a direction of a circumduction motion (step S102). Specifically, as described above, the calculation unit 292 calculates a deviation in a right-left direction with respect to a trace of a foot calculated from sensing data.

Next, the determination unit 293 determines which of a right foot and a left foot is equipped with the sensor device 100 (step S103).

According to the control of the determination unit 293, the display 220 displays a determination result (step S104). For example, when the determination unit 293 determines an inappropriateness of attaching the sensor device 100 to either a right foot or a left foot, as described above, the display 220 may display an alarm message.

After the step S104, the information processing device 200 exits the procedure of FIG. 5.

It is possible to form the sensor device 100 in a specific shape which may allow the sensor device 100 to be attached in an appropriate direction.

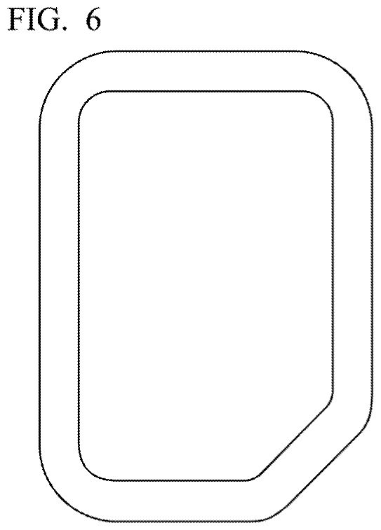

FIG. 6 is a bottom view showing a shape example of the sensor device 100 in view of its lower face. The lower face of the sensor device 100 serves as a ground-side face of the sensor device 100 when attached to a shoe in an appropriate direction.

In an example of FIG. 6, the sensor device 100 has a longitudinal length and a lateral length which differ from each other. In this illustration, the longitudinal length lies in a vertical direction of FIG. 6. For example, the sensor device 100 may have a longitudinal length of 40 milli-meters and a lateral length of 29 milli-meters. A hole adapted to the shape of the sensor device 100 is formed in each of the shoes 810. When putting the sensor device 100 into the hole formed in each of the shoes 810, it is possible to prevent a user from erroneously putting the sensor device 100 into its corresponding hole in a wrong direction of either a longitudinal direction or a lateral direction due to a difference of the longitudinal length and the lateral length.

FIG. 6 shows a slanted notch formed in the lower-right portion of the sensor device 100. When the sensor device 100 is put into a hole adapted to the shape of the sensor device 100 and formed in each of the shoes 810, it is expected that a user can put the sensor device 100 in its corresponding hole in an appropriate direction, i.e., a forward-backward direction and a rightward-leftward direction, due to the slanted notch.

It is possible to provide the lower face of the sensor device 100 shown in FIG. 6 with an indication requesting a user to attach the sensor device 100 to its corresponding shoe with its lower face directed downwardly. As described above, a user may attach the sensor device 100 to its corresponding shoe such that the sensor device 100 should be engaged with a hole of the shoe.

In addition, it is possible to provide the lower face of the sensor device 100 shown in FIG. 6 with an indication instructing a user to attach the sensor device 100 to either a left-side shoe or a right-side shoe.

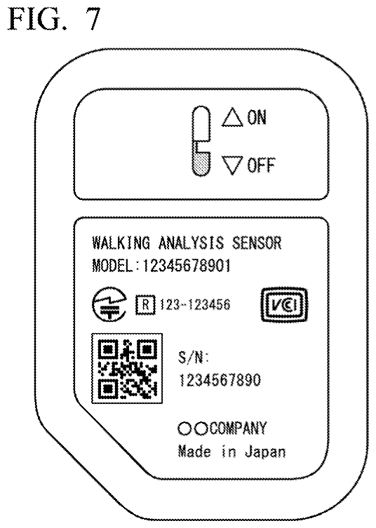

FIG. 7 shows an upper view of a shape example of the sensor device 100 in a view of the upper face of the sensor device 100. The upper face of the sensor device 100 serves as an exterior-side of the sensor device 100 when attached to its corresponding shoe in an appropriate direction.

The shape example of the sensor device 100 of FIG. 7 may correspond to a turnover of the shape example of the sensor device 100 of FIG. 6.

As shown in FIGS. 6, 7 in which the sensor device 100 has an upper-face indication and a lower-face indication which differ from each other, it is possible to reduce a possible error in which a user might erroneously attach the sensor device 100 to its corresponding shoe in a vertically-reverse manner.

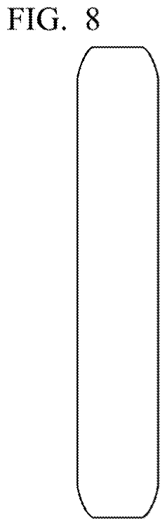

FIG. 8 is a side view showing a shape example of the sensor device 100 in a view of the side face of the sensor device 100.

In the example of FIG. 8, it is possible to set the thickness of the sensor device 100 to 7 milli-meters. By reducing the thickness of the sensor device 100 to be relatively thinner, it is possible to reduce a possible sense of incongruity when a target person wears the shoes 810 equipped with the sensor devices 100.

As described above, the acquisition unit 291 is configured to acquire sensing data from the sensor devices 100. The determination unit 293 is configured to determine an attached state of the sensor device 100 to its corresponding foot based on a motion of the foot to be calculated using sensing data.

As described above, the information processing device 200 is configured to determine an attached state of the sensor device 100 to its corresponding foot. In addition, the information processing device 200 is configured to determine whether an attached state of the sensor device 100 is appropriate or inappropriate. Upon determining an inappropriately-attached state of the sensor device 100, the information processing device 200 is configured to request a user (e.g., a target person) to fix an attached state of the sensor device 100. Alternatively, the information processing device 200 is advantageous in that it is possible to introduce a measure such as setting coordinates for analyzing sensing data depending on the attached state of the sensor device 100.

The calculation unit 292 is configured to calculate a motion of a foot on a walk using sensing data from the sensor device 100.

The information processing device 200 is designed to determine an attached state of the sensor device 100 by comparing its sensing data with a traveling direction of walking. With reference to acceleration components of sensing data in a forward-backward direction, for example, it is possible to determine whether or not a forward direction of the sensor device 100 matches a forward direction of walking.

The calculation unit 292 is configured to calculate a trace of a foot on a walk based on acceleration of the foot on a walk indicated by the sensing data of the sensor device 100.

The information processing device 200 is designed to determine a directivity of the sensor device 100 according to the calculated trace of a foot. For example, it is possible to determine a directivity of a foot's trace as a forward direction. In addition, the information processing device 200 is designed to determine which of a right foot and a left foot is equipped with the sensor device 100 according to a directivity of a circumduction motion described above.

The determination unit 293 is configured to determine an appropriateness with respect to an attached state of the sensor device 100.

Upon determining an inappropriately-attached state of the sensor device 100, the information processing device 200 may invoke some measures such as outputting an alarm or displaying a message requesting a user to re-fix the sensor device 100 to its corresponding shoe or the like.

When the determination unit 293 determines an inappropriately-attached state of the sensor device 100, the display 220 may display an alarm message.

The information processing device 200 is designed to notify a user (e.g., a target person) of a possible inappropriateness of sensing data and calculation results produced by the information processing device 200. In addition, the information processing device 200 may request a user to appropriately re-fix the sensor device 100 to its corresponding shoe by outputting an alarm such as displaying an alarm message.

In this connection, the calculation unit 292 may calculate a foot's motion including an amount of shifting of the foot in a lateral direction compared to a traveling direction of walking.

The information processing device 200 is designed to determine which of a right foot and a left foot is equipped with the sensor device 100 according to whether the foot is shifted rightwards or leftwards.

The coordinates-system-setting unit 294 is configured to set a coordinates system used to translate sensing data from the sensor device 100 according to an attached state of a foot to the sensor device 100.

Accordingly, it is expected that the information processing device 200 can appropriately process sensing data to produce an appropriate calculation result.

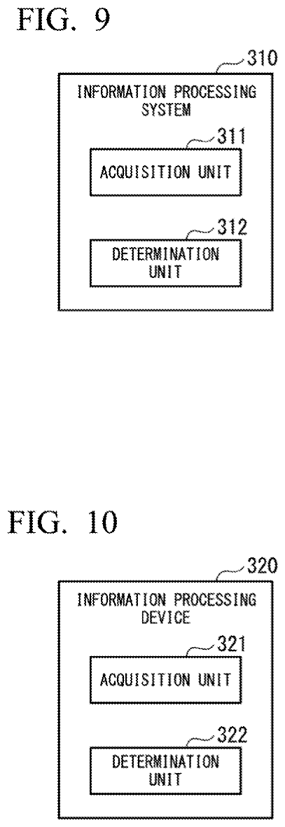

FIG. 9 is a block diagram showing a configuration example of an information processing system according to another exemplary embodiment. FIG. 9 shows an information processing system 310 including an acquisition unit 311 and a determination unit 312.

According to the above configuration, the acquisition unit 311 is configured to acquire sensing data of a sensor device attached to a foot of a target person. The determination unit 312 is configured to determine an attached state of the foot to the sensor device according to a foot's motion calculated using sensing data.

The information processing system 310 is designed to determine an attached state of a foot to a sensor device. In addition, the information processing system 310 is designed to determine whether an attached state of a sensor device to a foot of a target person is appropriate or not. Upon determining an inappropriately-attached state of a sensor device, the information processing system 310 may request a user to fix the attached state of a sensor device. Alternatively, the information processing system 310 may invoke some measures such as setting coordinates used to translate sensing data depending on the attached state of a sensor device to a foot of a target person.

FIG. 10 is a block diagram showing a configuration example of an information processing device according to another exemplary embodiment. FIG. 10 shows an information processing device 320 including an acquisition unit 321 and a determination unit 322.

According to the above configuration, the acquisition unit 321 is configured to acquire sensing data of a sensor device attached to a foot of a target person. The determination unit 322 is configured to determine an attached state of a sensor device to a foot according to a foot's motion calculated using sensing data.

The information processing device 320 is designed to determine an attached state of a sensor device to a foot of a target person. In addition, the information processing device 320 is designed to determine whether an attached state of a sensor device to a foot is appropriate or not. Upon determining an inappropriately-attached state of a sensor device, the information processing device 320 may request a user to fix an attached state of a sensor device. Alternatively, the information processing device 320 may invoke some measures such as setting coordinates used to translate sensing data depending on an attached state of a sensor device to a foot.

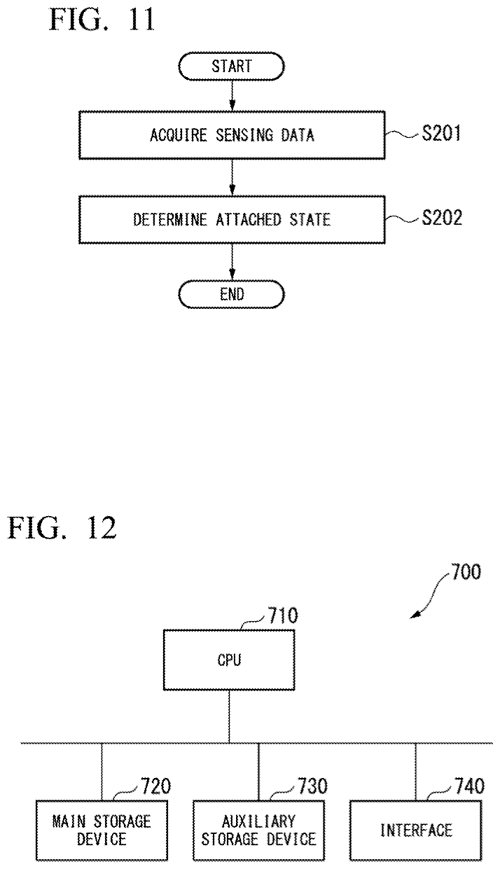

FIG. 11 is a flowchart showing an example of a procedure for an information processing method according to another exemplary embodiment. FIG. 11 shows an information processing method including a process for acquiring sensing data from a sensor device attached to a foot (step S201) and a process for determining an attached state of a sensor device to a foot according to a foot's motion calculated using sensing data (step S202).

According to the information processing method of FIG. 11, it is possible to determine an attached state of a sensor device to a foot. According to the information processing method of FIG. 11, it is possible to determine whether an attached state of a sensor device to a foot is appropriate or not. Upon determining an inappropriately-attached state of a sensor device, it is possible to request a user to fix an attached state of a sensor device. According to the information processing method of FIG. 11, it is possible to take some measures such as setting coordinates used to translate sensing data depending on an attached state of a sensor device to a foot.

FIG. 12 is a block diagram showing a configuration of a computer according to at least one exemplary embodiment.

According to the configuration shown in FIG. 12, a computer 700 includes a CPU 710, a main storage device 720, an auxiliary storage device 730, and an interface 740.

Any one of the information processing device 200, the information processing system 310, and the information processing device 320 may be implemented by the computer 700. In this case, the foregoing operations of processing parts can be organized into programs to be stored on the auxiliary storage device 730. The CPU 710 reads programs from the auxiliary storage device 730 so as to unwind programs on the main storage device 720, thus executing the foregoing processes according to programs. According to programs, the CPU 710 may secure storage areas for the foregoing storage units on the main storage device 720. The interface 740 having a communication function may conduct communication under the control of the CPU 710, thus achieving communication between each device and its counterpart device.

When the computer 700 is used to implement the information processing device 200, the control unit 290 and operations of its internal parts can be organized into programs to be stored on the auxiliary storage device 730. The CPU 710 may read programs from the auxiliary storage device 730 so as to unwind programs on the main storage device 720, thus achieving the foregoing functions according to programs.

According to programs, the CPU 710 may secure a storage area for the storage unit 280 on the main storage device 720. The interface 740 having a communication function may conduct communication under the control of the CPU 710, thus achieving communication of the communication unit 210. In addition, the interface 740 may display images on a display screen thereof under the control of the CPU 710, thus achieving a function of the display 220. Moreover, the interface 740 has an input device to receive a user operation, thus achieving a function of the operation-input unit 230.

When the computer 700 is used to implement the information processing system 310, the operations of the acquisition unit 311 and the determination unit 312 can be organized into programs to be stored on the auxiliary storage device 730. The CPU 710 may read programs from the auxiliary storage device 730 to unwind programs on the main storage device 720, thus achieving the aforementioned functions according to programs.

When the computer 700 is used to implement the information processing device 320, the operations of the acquisition unit 321 and the determination unit 322 can be organized into programs to be stored on the auxiliary storage device 730. The CPU 710 may read programs from the auxiliary storage device 730 to unwind programs on the main storage device 720, thus achieving the foregoing processes according to programs.

In this connection, it is possible to store programs, which may realize the entirety or part of the functions of the information processing device 200, the information processing system 310, and the information processing device 320, on computer-readable storage media, and therefore a computer system may load programs stored on storage media so as to execute programs, thus achieving the foregoing processes implemented by various parts. Herein, the term “computer system” may embrace operating systems (OS) and hardware such as peripheral devices.

The term “computer-readable storage media” refers to flexible disks, magneto-optical disks, ROM, portable media such as CD-ROM, and storage devices such as hard disks built in computer systems. The aforementioned programs may achieve some of the foregoing functions or may be combined with pre-installed programs of computer systems so as to achieve the foregoing functions.

Heretofore, the exemplary embodiments of the present invention have been described in detail with reference to the drawings; however, concrete configurations should not be limited to the foregoing embodiments, and therefore the present invention may include any design changes without departing from the subject matter of the invention.

### INDUSTRIAL APPLICABILITY

The exemplary embodiments of the present invention can be applied to information processing systems, information processing devices, information processing methods, and storage media.

### REFERENCE SIGNS LIST

1, 310 information processing system
100 sensor device
200, 320 information processing device
210 communication unit
220 display
230 operation-input unit
280 storage unit
290 control unit
291, 311, 321 acquisition unit
292 calculation unit
293, 312, 322 determination unit
294 coordinates-system-setting unit
811 left-foot shoe
812 right-foot shoe

What is claimed is:

1. An information processing system, comprising:
an acquisition unit configured to acquire sensing data from a sensor device used for a foot; and
a determination unit configured to determine an attached state of the sensor device to the foot according to a motion of the foot calculated using the sensing data.

2. The information processing system according to claim 1, further comprising a calculation unit configured to calculate the motion of the foot on a walk using the sensing data.

3. The information processing system according to claim 2, wherein the calculation unit is configured to calculate a trace of the foot on the walk based on acceleration of the foot on the walk indicated by the sensing data.

4. The information processing system according to claim 1, wherein the determination unit is configured to determine an appropriateness with respect to the attached state of the sensor device.

5. The information processing system according to claim 4, further comprising an alarm output unit configured to output an alarm when the determination unit determines that the attached state of the sensor device is inappropriate.

6. The information processing system according to claim 1, wherein the motion of the foot includes a shift of the foot in a lateral direction compared to a traveling direction of walking.

7. The information processing system according to claim 1, further comprising a coordinates-system-setting unit configured to set a coordinates system used to translate the sensing data according to the attached state of the sensor device to the foot.

8. An information processing device, comprising:
an acquisition unit configured to acquire sensing data from a sensor device used for a foot; and
a determination unit configured to determine an attached state of a sensor device to the foot based on a motion of the foot calculated using the sensing data.

9. An information processing method, comprising:
acquiring sensing data from a sensor device used for a foot; and
determining an attached state of the sensor device to the foot according to a motion of the foot calculated using the sensing data.

10. A non-transitory computer-readable storage medium configured to store a program causing a computer to execute:
acquiring sensing data from a sensor device used for a foot; and
determining an attached state of the sensor device to the foot according to a motion of the foot calculated using the sensing data.
